(19) United States
(12) Patent Application Publication
(10) Pub. No.: US 2014/0162977 A1
(43) Pub. Date: Jun. 12, 2014

(54) PROCESS AND DRESSING

(71) Applicant: Christian Stephenson, Wilmslow (GB)

(72) Inventor: Christian Stephenson, Wilmslow (GB)

(73) Assignee: Medtrade Products Limited, Crewe, Cheshire (GB)

(21) Appl. No.: 14/117,179

(22) PCT Filed: May 10, 2012
(86) PCT No.: PCT/EP12/58666

§ 371 Date: Feb. 24, 2014

(30) Foreign Application Priority Data
May 12, 2011 (GB) 1107929.0

(51) Int. Cl. A61L 15/28; C08B 37/08

(52) U.S. Cl. CPC A61L 15/28; C08B 37/003
USPC 514/55; 536/20; 204/157.64; 442/327

(57) ABSTRACT

An anti-microbial absorbent chitosan, optionally in the form or fibres, derivatised by treatment with high energy, in particular gamma, radiation, a non-woven fabric and an absorbent device comprising the derivatised chitosan, methods for the preparation of such a chitosan, non-woven fabric and an absorbent device, and the use of said absorbent device in wound care.

The present invention relates to an anti-microbial component of a absorbent device, to a method for the preparation of such an component, and in particular to a method for the preparation of a novel hydrophilic, anti-microbial derivatised chitosan, including in the form or fibres, an absorbent device comprising the component, a method for the preparation of such an absorbent device, and the use of said absorbent device.

The term ‘absorbent medical device’ as used herein includes wound dressings for acute wounds, including surgical wounds, and chronic and burn wounds, ostomy devices, surgical and dental sponges, and absorbent pads for the personal care sector, particularly for disposable sanitary devices such as nappies (diapers), disposable nappies and training pants, feminine care products, for example, tampons, sanitary towels, or napkins and pant liners, and incontinence products. It in particular includes (but is not limited to) an absorbent medical device comprising as an absorbent material, a water-insoluble organic or inorganic acyl substituted hydrocarbyl chitosan.

The term ‘organic or inorganic acyl’ as used herein includes organic acid residues such as carboxyl and inorganic acid residues such as sulphonyl.

The term ‘salified derivatised chitosan’ as used herein refers to an at least partially O-hydroxy-(organic or inorganic acyl)-hydrocarbylated chitosan, salified at a majority of the O-hydroxy-(organic or inorganic acyl)-groups in any positions in the glucosamine unit of the chitosan. Typical examples include salified carboxymethylated and sulphonatomethylated chitosans.

The salified derivatised chitosan may be produced by a process which comprises the reaction of a chitosan with a hydrocarbyl oxo acid or a salt thereof which is substituted by a nucleophilic leaving group, such as halo, in particular chloro in the presence of a base in an aqueous or non-aqueous medium.

The O-hydroxy-(organic or inorganic acyl)-hydrocarbylated chitosan product is believed to be one in which
a) one or both of the two types hydroxyl groups on the D-glucosamine units of the chitosan are converted at least in part to salified O—R—O— groups, where O—R is a hydrocarbyl oxoacid anion, e.g. carboxy- or sulphonato-methyl anion, i.e. to salified oxyhydrocarbyl oxoacid groups, for example salified carboxymethoxy or sulphonatomethoxy groups,
b) the remaining hydroxyl groups on the D-glucosamine units of the chitosan may be converted at least in part to hydroxyl group derivatised by the base to alkoxide groups (alkalisation), and
c) the amine groups on the D-glucosamine units of the chitosan may be converted at least in part to salified O—R—NH— groups, where O—R is a hydrocarbyl oxoacid anion as defined above. (c.f. the O—, N-carboxymethylated chitosans of CA 1 274 507.]

Depending on the preparative process used, the degree of N-substitution as in c) is generally considerably smaller that that of O-substitution, and in some cases may be negligible. However, reference herein to a salified derivatised (O-hydroxy-(organic or inorganic acyl)-hydrocarbylated) chitosan includes a reference to derivatised chitosans which are amine substituted as in c) above.

Either hydroxyl substitution may take place at any relevant hydroxyl position in the chitosan macromolecule, in any distribution up to the maximum degree of substitution that is possible.

Such salts of the derivatised chitosan at the O-hydroxy-(organic or inorganic acyl)-groups, may suitably be with any cation, but often with a pharmaceutically acceptable metal cation, usually an alkali metal cation. The chitosan is frequently derivatised in the above process by etherifying the chitosan hydroxyl groups by reaction with the sodium salt of the relevant hydrocarbyl oxoacid, such as chloroacetic or chloromethanesulphonic acid, in the presence of sodium hydroxide to catalyse the reaction, so that the cation is generally a sodium cation.

The maximum degree of salification of the acid groups in derivatised chitosans that is possible is 100%, and salified derivatised chitosan of the prior art generally have a degree of salification of at least 85%, for example 90%. They are commonly produced by a process in which a chitosan is derivatised by etherifying the chitosan hydroxyl groups by reaction with the sodium salt of the relevant hydrocarbyl oxoacid.

As described in our copending application PCT/EP 2012/050376 a particularly advantageous salified O-hydroxy-(organic or inorganic acyl)-hydrocarbylated chitosan may be produced by such a process, when carried out at a temperature below 50° C., preferably between 25 and 45° C., in particular between about 30 and about 40° C. It is believed that under such process conditions the degree of N-substitution is negligible.

Again, either hydroxyl substitution may take place at either hydroxyl position in the chitosan macromolecule, in any distribution up to the maximum degree of substitution that is possible.

The term ‘average degree of substitution’ in relation to a derivatised chitosan as defined refers to the mean number of glucosamine hydroxyl groups in all positions in the glucosamine unit converted to oxyhydrocarbyl oxoacid groups. These may be for example carboxymethoxy or sulphonatomethoxy groups.

The maximum degree of substitution is 2, when the D-glucosamine unit is substituted at both hydroxyl positions, and the degree of substitution when an average of one hydroxyl group is converted per D-glucosamine unit is 1.

Reference to “hydrocarbyl” or “hydrocarbylated” in relation to a chitosan, or a derivatised chitosan as defined, includes a reference respectively to “optionally substituted hydrocarbyl” and “optionally substituted hydrocarbylated”. Typical examples of such substituents include alkoxy, carboxy and sulphonate. The hydrocarbyl residues in the present hydrocarbylated chitosan salts are however often unsubstituted hydrocarbyl.

Reference herein to the “absorbency” of derivatised chitosan fibres herein refers to the absorbency of an, often non-woven, fabric or absorbent device comprising them, is to the capacity of the derivatised chitosan fibres to take up fluid. The fluid is absorbed between the fibres and into the internal fibre structure and the fibre swells. Measurement of the overall absorptive capacity of an absorbent material or of a absorbent device, in particular a medical absorbent device, comprising such material is a convenient and effective process for determining the effectiveness of the absorbent material or device for absorbent applications, such as wound dressings.

By “substantially water-insoluble” in relation to a material, such as a chitosan or a derivatised chitosan as defined, is meant herein that when the material is exposed to an excess of an aqueous medium it does not dissolve into solution, or at least that dissolution is so low as to have no significant effect on the physical properties of the polymer.

The term ‘high energy radiation’ as used herein includes subatomic particles or electromagnetic waves (photons) with energies above a few electron volts (eV) that are energetic enough to detach electrons from atoms or molecules, thus ionizing them to produce free radicals containing unpaired electrons, and includes charged particles such as electrons, positrons and alpha particles; plasmas; neutrons; and high frequency ultraviolet, x-rays, and gamma rays.

The term ‘irradiated derivatised chitosan’ as used herein refers to a derivatised chitosan which has been subjected to high energy irradiation.

No statements made on belief herein, in particular as to the nature, physical form and physical properties of any chitosan product, and of any fabric or absorbent devices, in particular any wound dressings comprising such chitosan product, or of any process for the manufacture thereof, shall be construed as in any way as limiting the scope of the present invention. It will also be appreciated that the composition of all derivatised chitosans may vary with the properties of the starting material chitosan from which it is derived, including its source, the degree of deacetylation of the chitin starting material that is reached in its production, and its molecular weight.

There is a need for (generally fibrous) absorbent materials, for example for wound dressings, that are substantially insoluble in the aqueous wound fluid, but are swellable and gellable to absorb the fluid, yet retain wet cohesion, so that they can be removed in one piece from the wound.

It is known to produce and process a partially carboxymethylated modified chitosan fibre to an absorbent non-woven fabric component of a wound dressing by fibre opening, web formation and needling the fibre finish on the surface of the chitosan.

Such fabric is often intended for fluid absorbance in dressings, in particular from acute wounds, including surgical wounds, and chronic and burn wounds.

A first technical problem of the prior art is that the high aqueous absorbency of the partially carboxymethylated chitosan fibre causes difficulty in fibre opening and web formation during subsequent processing of partially carboxymethylated chitosan amine salt fibre to a non-woven fabric by a non-woven technique.

A second technical problem of the prior art is that after being processed by a non-woven processing machine, the fibre finish on the surface of the partially carboxymethylated chitosan fibre makes the non-woven fabric hydrophobic.

The hydrophobicity adversely affects the fluid absorbency of the dressings of the prior art, in particular from acute wounds, including surgical wounds, and chronic and burn wounds.

To be useful in an absorbent device, the fibres of an absorbent material may suitably have an absorbency of 8 to 30 grams per gram (g/g) standard solution, preferably 12 to 27 g/g, and more preferably 16 to 23 g/g.

It is therefore an object of the present invention to provide a therapeutically active derivatised chitosan fibre which is not only readily processable in fibre opening and web formation during subsequent processing to a non-woven fabric component of such a wound dressing, and such a dressing component which has a wound-facing surface which is not hydrophobic and thus has good absorbency of fluids, in particular from acute wounds, including surgical wounds, and chronic and burn wounds, and thus avoids the disadvantage of the hydrophobicity of non-woven fabric made from partially carboxymethylated chitosan amine salt fibre.

It is also an object of the present invention in overcoming the disadvantage of the hydrophobicity of prior art fabrics to provide a therapeutically active derivatised chitosan fibre which meets the above criteria for adequate and good absorbency of fluids, in absorbent devices, and in particular in advance wound dressings.

Surprisingly, we have now found that the processability of a derivatised chitosan may be improved, and the absorbency of the derivatised chitosan and of an absorbent device comprising it may be improved by treatment with high energy radiation. More surprisingly, we have now found that it is not necessary to treat the irradiated product with a liquid hydrophilic agent to increase its absorbency.

Most surprisingly, the absorbency of the derivatised chitosan or of an absorbent device comprising it may be improved to a large and unexpected degree by treatment with high energy radiation.

Thus, in order to solve the above technical problems, according to a first aspect of the present invention, there is provided an irradiated derivatised chitosan as defined.

That is a chitosan, at least partially substituted at the D-glucosamine hydroxyl positions by salified O-hydroxy-(organic or inorganic acyl)-hydrocarbyl groups which has been subjected to high energy radiation. Examples of such O-hydroxy-(organic or inorganic acyl)-hydrocarbyl groups include carboxy- or sulphonato-methyl.

The product is preferably in the form of fibres, and the specific form of the fibres will often be determined by the specific from of the starting material of the process of the second aspect of the present invention.

The irradiated derivatised (O-hydroxy-(organic or inorganic acyl)-hydrocarbylated) chitosans of the invention are highly advantageous for use as absorbent materials in absorbent devices because they are substantially water-insoluble, non-toxic and odourless. They also have antimicrobial (including antibacterial and antimycotic), antiphlogistic, haemostatic, deodorant and antalgic capability, and facilitate wound healing.

The present irradiated derivatised chitosans may be fibres which may be formed into, for example non-woven, fabrics, for example for wound dressings,
a) which swell in contact with water to become an elastic gel material, and exhibit good absorption and retention of fluid and are insoluble, and
b) where the fibres have sufficient dry strength to be processed into a, for example, non-woven, fabric, and when in a wound dressing, the fibres and the fabric have sufficient wet strength and cohesion, that the fibres and fabric can be removed in one piece from a wound to which they are applied, without irrigation, and with minimum pain and shedding. Absorption of fluid is virtually instantaneous since ionic exchange is not required for the fibres to become gellable.

The therapeutically active, irradiated derivatised chitosan fibres of the present invention meet the criteria for adequate and good absorbency of fluids in absorbent devices, and in particular in advanced wound dressings. The irradiated derivatised chitosan fibres may suitably have an absorbency of 8 to 30 grams per gram (g/g) of saline solution, often 12 to 27 g/g, and more often 16 to 23 g/g.

The pre-absorption irradiated derivatised chitosan fibres according to the present invention may have a monofilament linear density of 0.1 to 30, preferably about 0.5 to 20, and more preferably 0.9 to 8, for example 1 3 to 5 decitex, and a strength of 0.8 to 2.2, such as 1 to 2, for example 1.2 to 1.8 cN/dtex.

However, the initial aqueous absorbency of the irradiated derivatised chitosan fibre is not so high that it causes difficulty in fibre opening and web formation during subsequent processing of the irradiated derivatised chitosan fibre to a non-woven fabric by a non-woven technique. The water-insoluble irradiated derivatised chitosans of the present invention are also advantageous compared to the prior art carboxymethyl chitosans because the absorptive capacity is not adversely affected by processing into absorbent device, for example wound dressing, non-woven components.

They are also affected to a lesser extent in use by changes in pH. Wound dressings containing these materials continue to absorb to a good level at low pH.

According to a second aspect of the present invention, there is also provided a process for producing an irradiated derivatised chitosan, which comprises treating a salified derivatised chitosan with high energy radiation.

As defined, in such a salified derivatised chitosan, one or both of the hydroxyl groups on the D-glucosamine units of the chitosan have been converted at least in part to salified O—R—O— groups as hereinbefore defined, and the other hydroxyl groups on the D-glucosamine units of the chitosan have optionally been converted to hydroxyl groups derivatised by a base (preferably with a pharmaceutically acceptable metal cation, usually an alkali metal cation.

As noted above, in the salified derivatised chitosan, the degree of substitution of the hydroxyl groups in all positions by M-O—R—O— groups is suitably less than 0.8, preferably less than 0.7, more preferably less than 0.6 for the derivatised chitosan to be substantially water-insoluble. The average degree of substitution in the derivatised chitosan polymer may suitably be from about 0.1 to about 0.4, for example from about 0.15 to about 0.35, such as from about 0.2 to 0.3. It is believed that the average degree of such substitution is unchanged in the irradiated derivatised chitosan polymer of the present invention.

The process is often carried out as a single step in one treatment vessel.

It will be clear to those skilled in the art that the form of the derivatised chitosan starting material of the process may have a significant effect on the form of the irradiated derivatised chitosan product. It will be appreciated that many forms or modifications of the starting material derivatised chitosan may of course be used to produce suitable products for use in absorbent, in particular wound care medical, devices, and these fall within the scope of the present invention.

Irradiation is preferably effected on a solid substrate, and preferably in the presence of water vapour. Moisture may be present in the starting material of, or introduced during, the preparation of the salified derivatised chitosan and/or the irradiation process.

It is believed that the high energy radiation serves as an energy source to initiate polymerisation and/or cross-linking. It will be clear to those skilled in the art that the physical form of the chitosan starting material, the desired physical properties of the irradiated product, and the nature, power and dosage of the radiation may have a significant effect on the necessary conditions of the irradiation process.

As noted above, high energy radiation as used herein includes charged particles such as electrons, positrons and alpha particles; plasmas; neutrons; and high frequency ultraviolet, x-rays, and gamma rays.

Generally, the higher the dosage (total energy input) of the radiation, the greater the degree of modification of the starting material derivatised chitosan (for example polymerisation and/or cross-linking. It will be clear to those skilled in the art that the nature of the radiation, for example its frequency, may also have a significant effect on the degree and rate of modification of the starting material derivatised chitosan in the irradiation process, and that both the frequency and the dosage should be chosen to avoid any deleterious effect on the substrate.

Where irradiation is (preferably) effected in the presence of water vapour, the product is often subsequently air-dried, in particular if it is to be subjected to further processing.

The salified derivatised chitosan may be prepared by a process which comprises contacting chitosan preferably as a fibre
(a) with a base in an aqueous or non-aqueous medium, and
(b) with a solution in an aqueous or non-aqueous medium of a salt of the formula (I):

M-O—R-T (I)
wherein
M is a Group IA metal cation;
O—R is a hydrocarbyl oxoacid anion; and
T is a nucleophilic leaving group; and
(c) isolating, washing and drying the product of steps (a) and (b),

Preferably all the steps (a) to (c) are carried out at a temperature below 50° C.

The derivatised chitosan fibre product from steps (a) to (c) of this process is believed to be one in which one or both of the hydroxyl groups on the D-glucosamine units of the chitosan are converted at least in part to salified O—R—O— groups (etherification).

The base is preferably an alkali metal hydroxide, in which case the metal cation is preferably the same as M in the compound of formula (I), preferably an alkali metal hydroxide, such as sodium hydroxide, and these hydroxyl functions are preferably converted to M-O—R—O— groups. The other hydroxyl groups on the D-glucosamine units of the chitosan may be generally converted to hydroxyl group derivatised by the base (alkalisation), preferably M-O— groups.

Either substitution may take place at either hydroxyl position in the chitosan macromolecule, in any distribution up to the maximum degree of substitution that is possible.

The relevant derivatised hydroxyl groups in all positions are described hereinafter as M-O—R—O— groups and M-O— groups on the D-glucosamine units in the derivatised chitosan polymer. However, the term ‘M-O—R—O— groups’ as used herein includes hydroxyl groups on the D-glucosamine units of the chitosan that have been converted to salified O—R—O— groups (etherification), being only preferably M-O—R—O— groups in which the metal cation is preferably the same as M in the compound of formula (I). The term ‘M-O— groups’ as used herein includes hydroxyl groups on the D-glucosamine units of the chitosan that have been converted to hydroxyl group derivatised by a base (alkalisation), being only preferably M-O— groups.

The average degree of substitution refers to the mean number of hydroxyl groups in all positions converted to M-O—R—O— groups, that is, the mean number of moles of M-O—R—O— groups per mole of D-glucosamine unit in the chitosan polymer. The maximum degree of substitution is therefore 2, when the D-glucosamine unit is substituted at both hydroxyl positions, and the degree of substitution when an average of one hydroxyl group is converted per D-glucosamine unit 1.

However, the average degree of substitution is suitably less than 0.8, preferably less than 0.7, more preferably less than 0.6 for the derivatised chitosan to be substantially water-insoluble.

The average degree of substitution in the derivatised chitosan polymer of the present invention may suitably be from about 0.1 to about 0.4, for example from about 0.15 to about 0.35, such as from about 0.2 to 0.3. In the salified derivatised chitosan intermediate:

M may suitably be a sodium or potassium cation, preferably a sodium cation.

O—R may suitably be a hydrocarbyl acylate of an organic or inorganic oxoacid, for example an alkane acylate of an organic or inorganic oxoacid.

O—R may thus suitably be, for example, an alkanoate, preferably a lower alkanoate with 2 to 6 carbon atoms, such as an acetate, glyoxylate, propionate, pyruvate or butyrate, preferably acetate, propionate or butyrate preferably attached in the 2-position to the chitosan oxy group.

The alkanoate moiety may be branched or unbranched, and hence suitable butyrates may be n-butyrate or iso-butyrate. The alkanoate group that is most preferred is acetate.

O—R may also suitably be, for example an alkanesulphonate, preferably a lower alkanesulphonate with 2 to 6 carbon atoms, such as a methanesulphonate, ethanesulphonate, or propanesulphonate, preferably attached in the 1-position to the chitosan oxy group, and more preferably methanesulphonate. The alkane moiety may be branched or unbranched, and hence suitable propane sulphonates may be propane-1- or 2-sulphonate, and butanesulphonates may be butane-1-sulphonate, 2,2-dimethylethane-1-sulphonate or 1,2-dimethylethane-1-sulphonate. The alkane sulphonate substituent group that is most preferred is methane sulphonate.

O—R may also suitably be an arenecarboxylate, such as a benzoate or toluate, substituted by a nucleophilic leaving group T.

O—R may also suitably be an arenesulphonate, such as benzenesulphonate or toluenesulphonate, substituted by a nucleophilic leaving group T.

Suitable and preferred nucleophilic leaving groups T, when O—R is an arenecarboxylate or an arenesulphonate, and suitable and preferred substitution positions for T will be well-known to the skilled person.

Process steps (a) to (c) in the present invention are a method for the preparation of a derivatised chitosan precursor of the irradiated derivatised chitosan polymer of the present invention, and these steps are described further in our copending application to process steps (a) to (c).

As noted above, the derivatised chitosan which is the product of reaction steps (a) to (c) above is believed to be one in which one or both of the hydroxyl groups on the D-glucosamine units of the chitosan are converted at least in part to salified O—R—O— groups (etherification), where O—R is a hydrocarbyl oxoacid anion.

In process steps (a) and (b), carried out at a temperature below 50° C., the base is preferably an alkali metal hydroxide, in which case the metal cation is preferably the same as M in the compound of formula (I), preferably an alkali metal hydroxide, such as sodium hydroxide, and these hydroxyl functions are preferably converted to M-O—R—O— groups; and the other hydroxyl groups on the D-glucosamine units of the chitosan are generally converted to hydroxyl group derivatised by the base (alkalisation), preferably M-O— groups. Either substitution may take place at either hydroxyl position in the chitosan macromolecule, in any distribution up to the maximum degree of substitution that is possible.

It will also be appreciated that the composition of the starting material derivatised chitosan may vary with the properties of its starting material chitosan, including its source, the degree of deacetylation of the chitin starting material that is reached in its production, and its molecular weight. The degree of deacetylation is often from 60 to 100%, and the average molecular weight is often between 3800 to 20,000 daltons.

All chitosan materials may be modified in the present process to produce absorbent products, for example for use in wound care medical devices. For example, a natural chitosan derived from shrimp or snow crab and/or a natural chitin may be utilised.

The derivatised chitosan starting material of steps (a) to (c) of the process may be used in the form of fibre tow.

The fibres may be used in a wide range of lengths, for example a few mm, such as 2 mm to 5 mm, to several tens of mm, for example 100 mm or more. It will be clear to those skilled in the art that the physical form of the chitosan starting material may have a significant effect on the physical form of the derivative chitosan product of the process.

Air-dried irradiated derivatised chitosan fibre of the first aspect of the invention may be converted into a non-woven fabric by fibre opening, web formation and needling, which may suitably be of 30 to 200 g/m² or more.

The irradiated derivatised chitosans of the present invention may be processed according to known methods into a wide variety of forms, depending on their intended use, for example as a non-woven component of an absorbent device.

The manner in which the derivative chitosan is processed has a significant effect on the properties of the final product, particularly the strength, gelling time, and absorbency.

A third aspect of the present invention thus provides an absorbent non-woven fabric comprising irradiated derivatised chitosan fibres of the first aspect of the invention.

As noted above, the overall absorptive capacity of the fabric is sensitive to the sizes and interconnectivity of the inter-fibre volumes within it, and hence to the parameters of the process for its manufacture, and will often differ from that of the component fibres. The therapeutically active, absorbent non-woven fabrics of the present invention comprising irradiated derivatised chitosan fibres meet the criteria for adequate and good absorbency of fluids in absorbent devices, and in particular in advanced wound dressings.

The non-woven fabrics comprising irradiated derivatised chitosan fibres may suitably have an absorbency of 8 to 30 grams per gram (g/g) of saline solution, often 12 to 27 g/g, and more often 16 to 23 g/g.

In many embodiments of non-woven fabrics comprising the water-insoluble irradiated derivatised chitosan according to the invention, for example for absorbent devices, it is the only absorbent material present. Such embodiments do not contain other absorbent materials such as hydrogels, anion-exchange resins, etc. or relatively non-absorbent, for example strengthening polymeric, materials

In other embodiments of non-woven fabrics comprising the water-insoluble irradiated derivatised chitosan according to the invention, for example for absorbent devices, however, other materials are present. Such embodiments may contain other absorbent materials such as hydrogels, anion-exchange resins, etc. or relatively non-absorbent, for example strengthening polymeric materials, often in the form of fibres which are intermingled with the irradiated derivatised chitosan according to the invention.

Thus, another embodiment of the present invention is directed to a non-woven fabric comprising an irradiated derivatised chitosan of the present invention which is reinforced with a reinforcing fibre blended with the water-insoluble irradiated derivatised chitosan.

Examples of other materials that may be present include other absorbent materials, such as hydrogels, for example an alginate, or anion-exchange resins, or a mixture thereof; or relatively non-absorbent, for example polymeric strengthening, materials, such as unmodified chitosan or thermoplastic bicomponent fibres, most preferably having a polyolefin component, for example comprising a polyolefin-containing polymeric material of which the largest part (by weight) consists of homo- or copolymers of monoolefins such as ethylene, propylene, 1-butene, 4-methyl-l-pentene, etc.

For example, un-modified chitosan fibre may be added, in a weight ratio of chitosan fibre to the derivatised chitosan fibre of 1:9 to 9:1, for example of 1:6 to 6:1 or 1:3 to 3:1.

It will be clear to those skilled in the art that the addition of unmodified chitosan to the derivatised chitosan material may have a significant improving effect on the strength of the material in a non-woven wound dressing.

The air-dried irradiated derivatised chitosan fibre including such additive materials may then be converted into a non-woven fabric as for derivatised chitosan fibre without such additive materials by fibre opening, web formation and needling, which may suitably be of 30 to 200 g/m² or more.

The absorbent non-woven fabric of the third aspect of the present invention may also be provided by an alternative process for its manufacture. This alternative process forms a fourth aspect of the present invention, and is a variant of the process according to the second aspect of the present invention for producing an irradiated derivatised chitosan.

In this alternative process, the salified derivatised chitosan staring material of the process of the second aspect of the invention (which may be the product of steps (a) to (c) of a process described hereinbefore, is processed according to known methods into a non-woven fabric, before being treated with high energy radiation.

Conditions for irradiation are similar to those described further hereinbefore in respect of the processes of the first aspect of the present invention for the high energy irradiation of a derivatised chitosan.

As noted above, in a non-woven fabric comprising the water-insoluble derivatised chitosan which is the process starting material, it may be the only absorbent material present.

In other non-woven fabrics comprising the water-insoluble derivatised chitosan which are the process starting materials, other materials may be present. Such embodiments may contain other absorbent materials such as hydrogels, anion-exchange resins, etc. or relatively non-absorbent, for example strengthening polymeric materials, often in the form of fibres which are intermingled with the irradiated derivatised chitosan according to the invention.

The manner in which the salified derivatised chitosan is first processed may have a significant effect on the properties of the final non-woven product of step (d), particularly the strength, gelling time, and absorbency.

As noted above, the overall absorptive capacity of such a fabric is sensitive to the sizes and interconnectivity of the inter-fibre volumes within it, and hence to the parameters of the process for its manufacture, and will often differ from that of the component fibres.

The starting material non-woven fabric for step (d) may be prepared from air-dried salified derivatised chitosan fibre (including any additive materials as above) by fibre opening, web formation and needling, which may suitably give rise to a non-woven of 30 to 200 g/m² or more.

The therapeutically active, absorbent non-woven fabrics produced by the process of the fourth aspect of the present invention comprising irradiated derivatised chitosan fibres also meet the criteria for adequate and good absorbency of fluids in absorbent devices, and in particular in advanced wound dressings. The non-woven fabrics comprising irradiated derivatised chitosan fibres may suitably have an absorbency of 8 to 30 grams per gram (g/g) of saline solution, often 12 to 27 g/g, and more often 16 to 23 g/g.

It will be clear to those skilled in the art that the area density of the product of the processing may have a significant effect on the absorbency of the processed irradiated derivatised chitosan material.

The non-woven fabric (prepared by either of the above routes) may then be converted to an anti-microbial absorptive component of a non-woven absorptive device by cutting, packaging and sterilising.

Their absorbent properties, biodegradability, and the fact that chitosan is a renewable material, mean that the irradiated derivatised chitosan absorbent materials of the present invention may be used in a wide range of absorbent devices.

In a fifth aspect of the present invention there is provided an absorbent device (as defined hereinbefore) comprising irradiated derivatised chitosan fibres of the second aspect of the invention.

As noted above, the overall absorptive capacity of the fabric in the device is sensitive to the sizes and interconnectivity of the inter-fibre volumes within it, and hence to the parameters of the process for its manufacture, and will often differ from that of the component fibres.

The therapeutically active, absorbent non-woven fabrics of the present invention comprising irradiated derivatised chitosan fibres meet the criteria for adequate and good absorbency of fluids in absorbent devices, and in particular in advanced wound dressings. The non-woven fabrics comprising irradiated derivatised chitosan fibres have an absorbency of 8 to 30 grams per gram (g/g) of saline solution, often 12 to 27 g/g, and more often 16 to 23 g/g.

In absorbent devices comprising non-woven fabrics comprising the water-insoluble derivatised chitosan which is the process starting material, it may be the only absorbent material present. In other non-woven fabrics comprising the water-insoluble derivatised chitosan which are the process starting materials, other materials may be present. Such embodiments may contain other absorbent materials such as hydrogels, anion-exchange resins, etc. or relatively non-absorbent, for example strengthening polymeric materials, often in the form of fibres which are intermingled with the irradiated derivatised chitosan according to the invention.

Their absorbent properties, biodegradability, and the fact that chitosan is a renewable material, mean that the irradiated derivatised chitosan absorbent materials of the present invention may be used in a wide range of absorbent devices.

The absorbent materials of the present invention exhibit instant gelling in aqueous media, good absorbency and, crucially, good retention of absorbency in an acidic environment.

When fully hydrated, the absorbent medical device is substantially transparent. This is advantageous in wound care applications since, if used in a dressing with a backing layer, or a window in a backing layer, which is transparent, the state of the underlying wound can be determined without removing the dressing. This renders them ideal for use as an absorbent wound care product, such as a dressing, or as part of an absorbent wound care product.

They are suited for use in wound dressings for acute wounds, including surgical wounds, and chronic and burn wounds. They are particularly useful for wounds with moderate to high levels of exudates, and for flat or cavity wounds of this type. Typical examples include burn wounds, and chronic wounds, such as pressure sores and leg ulcers.

The dressing, when covering a wound, is able to prevent water in body fluids from being lost, providing a favourable moist environment for wound healing and maintaining a fluid-free, maceration-free, germ-free wound surface.

They may also be used in ostomy devices, and surgical and dental sponges.

Thus, one embodiment of this fifth aspect of the present invention provides an absorbent medical device comprising the irradiated derivatised chitosan fibres of the first aspect of the invention.

Their absorbent properties, biodegradability, and the fact that chitosan is a renewable material, mean that the irradiated derivatised chitosan absorbent materials of the present invention may be used in a wide range of absorbent devices.

Preferred irradiated derivatised chitosan products for use in wound care medical devices are carded, needle-bonded non-wovens.

The irradiated derivatised chitosan which is comprised in a non-woven fabric, in turn comprised in an absorbent device or absorbent component thereof, as described further hereinbefore, may suitable be present as about 10 to 13%) of the absorbent medical device. They preferably have a pre-absorption monofilament linear density of 0.1 to 30, preferably about 0.5 to 20, and more preferably 0.9 to 8, for example 1 3 to 5 decitex, and a strength of 0.8 to 2.2, such as 1 to 2, for example 1.2 to 1.8 cN/dtex.

Whilst they swell in contact with water to become an elastic gel material, and exhibit good absorption and retention of fluid, they maintaining their integrity sufficiently, for example in wound dressings, to be removed from the wound site in one piece, without irrigation, and with minimum pain and shedding.

Absorption of fluid is virtually instantaneous since ionic exchange is not required for the fibres to become gellable.

The use of the absorbent materials of the present invention is not limited to medical products, however, and they are useful for other applications.

Their absorbent properties, biodegradability, and the fact that chitosan is a renewable material, mean that absorbent devices comprising the irradiated derivatised chitosans of the invention are also particularly desirable for use in absorbent pads for the personal care sector, particularly for disposable sanitary devices such as nappies (diapers), disposable nappies and training pants, feminine care products, for example, tampons, sanitary towels, or napkins and pant liners, and incontinence products.

Thus, another embodiment of this fifth aspect of the present invention provides an absorbent personal care device comprising the irradiated derivatised chitosan fibres of the second aspect of the invention.

The absorbent device of the fifth aspect of the present invention may also be provided by an alternative process for its manufacture, which forms a sixth aspect of the present invention. This process is a variant of the process according to the second aspect of the present invention for producing an irradiated derivatised chitosan.

In this variant, the salified derivatised chitosan starting material of the process of the second aspect of the invention, which may be the product of the steps (a) to (c) process described hereinbefore, or starting material of the second process, is processed according to known methods into a non-woven fabric, which is in turn processed according to known methods into an absorbent device or absorbent component thereof, before being treated with high energy radiation.

Conditions for irradiation are similar to those described further hereinbefore in respect of the process of the first aspect of the present invention for the high energy irradiation of a derivatised chitosan.

In a seventh aspect, the invention provides a method of treating a human or animal body with a device according to the fifth aspect of the invention.

The invention will now be illustrated by the following non-limiting examples.

### EXAMPLE 1

This example demonstrates steps (a) to (c) of the process for making salified derivatised chitosan fibres by carboxymethylation of the chitosan fibres in a solution containing 10.625 kg sodium hydroxide (40% solution), 4.5 kg sodium chloroacetate (44% solution) and 100 kg ethanol (60% solution) was prepared, the remainder being deionised water. The solution was heated to 30° C. 5.0 kg chitosan fibres were added and the solution reacted for 45 minutes. The temperature of the solution was then increased to 40° C., and the fibres were reacted for a further 120 minutes.

The modified fibres underwent 5 spin dry and ethanol wash cycles and a final spin dry. The fibres were treated with a spin finish comprised 1.523% Tween 20 and 98.478% ethanol (95%). The fibres underwent another spin dry after fibre finish application and were left to air dry and condition.

### EXAMPLE 2

The product derivatised chitosan fibres of Example 1 were processed according to known methods on a non-woven processing machine into a non-woven fabric, which is suitable for use as an anti-microbial absorptive component of a non-woven absorptive device, such as a wound dressing.

### EXAMPLE 3

The product of Example 2 was irradiated at 35 kGy.

### EXAMPLE 4

The gram per gram absorbency of the fabric of Example 3 before and after gamma irradiation was determined as follows:

Three fabric specimens were cut to 5 cm×5 cm (2″ by 2″) (25 cm²). Each specimen was weighed.

Each specimen was then placed in a Petri dish and covered with an excess of Solution A. Solution A is a specific solution of sodium chloride and calcium chloride QCP090 as used in British/European Standard BS EN 13726-1:2002 Test methods for primary wound dressings Part 1 Aspect of absorbency.) Each specimen was left in the Solution A for 180 seconds then removed from the Solution A by taking one corner with the forceps, and allowed to drain for 15 seconds. Each specimen was then weighed.

Results before and after irradiation: respectively were 17 g/g and 20 g/g.

1. An irradiated derivatised chitosan, which is a chitosan, at least partially substituted at the D-glucosamine hydroxyl positions by salified O-hydroxy-(organic or inorganic acyl)-hydrocarbyl groups which has been subjected to high energy radiation.

2. An irradiated derivatised chitosan according to claim 1 in which the O-hydroxy-(organic or inorganic acyl)-hydrocarbyl groups are carboxymethyl or sulphonato-methyl.

3. An irradiated derivatised chitosan according to claim 1 in the form of fibres.

4. A process for producing an irradiated derivatised chitosan according to claim 1, which comprises treating a salified derivatised chitosan, in which chitosan hydroxyl groups have been converted at least in part to salified O—R—O— groups wherein R—O is a hydrocarbyl oxoacid anion, with high energy radiation

5. A process according to claim 4, which is carried out with gamma rays.

6. A process according to claim 4, wherein the salified derivatised chitosan is irradiated at 20-40 kGy.

7. A process according to claim 4 wherein the moiety —O—R—O is carboxymethyl anion.

8. A process according to claim 4, wherein the moiety —0—R-T is sulphonylmethyl anion.

9. A process according to claim 4 wherein the salified derivatised chitosan is produced by a process which comprises contacting chitosan fibre
(a) with a base in an aqueous or non-aqueous medium, and
(b) with a solution in an aqueous or non-aqueous medium of a salt of the formula (I):
M-O—R-T (I)
wherein
M is a Group IA metal cation;
O—R is a hydrocarbyl oxoacid anion; and
T is a nucleophilic leaving group; and
(c) isolating, washing and drying the product of steps (a) and (b), all at a temperature below 50° C.

10. An absorbent non-woven fabric comprising an irradiated derivatised chitosan according to claim 1.

11. A process for preparing a non-woven fabric according to claim 10, which comprises treating a non-woven fabric comprising a salified derivatised chitosan, in which the hydroxyl groups have been converted at least in part to salified O—R—O— groups wherein R—O is a hydrocarbyl oxoacid anion, with high energy radiation.

12. An absorbent device comprising an irradiated derivatised chitosan according to claim 1.

13. A process for preparing an absorbent device according to claim 12, which comprises treating an absorbent device comprising a salified derivatised chitosan, in which the hydroxyl groups have been converted at least in part to salified O—R—O— groups, with high energy radiation.

14. The use of an absorbent device according to claim 13 in wound care.
